(19) United States
(12) Patent Application Publication
(10) Pub. No.: US 2024/0197764 A1
(43) Pub. Date: Jun. 20, 2024

(54) METHOD FOR INHIBITING BODY FAT ACCUMULATION AND METHOD FOR INHIBITING BODY WEIGHT GAIN

(71) Applicant: NAGAOKA CO., LTD., Osaka (JP)

(72) Inventors: Keiichiro SUGIMOTO, Osaka (JP); Hiroyuki FUJISAWA, Tokyo (JP); Kazuya NAKAGAWA, Osaka (JP)

(73) Assignee: NAGAOKA CO., LTD., Osaka (JP)

(21) Appl. No.: 18/504,922

(22) Filed: Nov. 8, 2023

(30) Foreign Application Priority Data
Nov. 9, 2022 (JP) 2022-179879

(51) Int. Cl. A61K 31/7032; A61K 36/61; A61P 3/06

(52) U.S. Cl. CPC A61K 31/7032; A61K 36/61; A61P 3/06

(57) ABSTRACT

A method for inhibiting body fat accumulation comprises a process of taking 3.38 mg or more of oenothein B indicated by the following formula (I) as an effective amount per day.

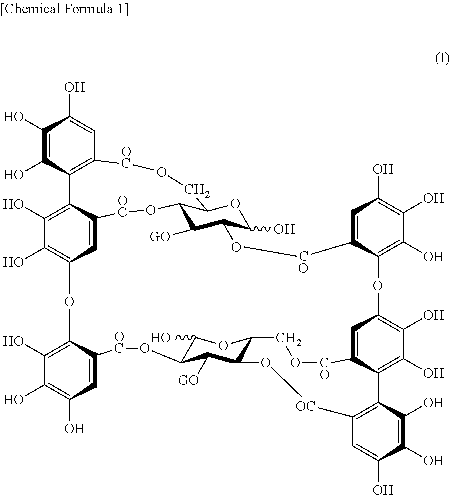

G in the formula (I) represents a structure of the following formula (II).

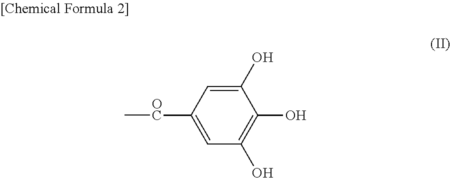

### TECHNICAL FIELD

The present disclosure relates to a method for inhibiting body fat accumulation and a method for inhibiting body weight gain.

### BACKGROUND ART

Surplus energy that is not consumed in daily life is accumulated as fat in a body due to excessive calories intake resulting from Westernization of food culture, overeating, or unbalanced eating. Fat accumulated in the body includes visceral fat, which accumulates around internal organs such as the stomach and intestines, liver fat, which accumulates in the liver, and subcutaneous fat, which accumulates in the subcutaneous tissue. Obesity is a state in which fat is accumulated in the body.

Obesity causes health disorders such as dyslipidemia, diabetes, hypertension, fatty liver and vascular disease. To reduce the risk of such health disorders, it is important to prevent obesity. Therefore, one possible way to prevent obesity is to prevent excess fat from accumulating in the body.

For example, Patent Document 1 describes a visceral fat reducing agent containing a grain culture of the Cordyceps militaris mycelium. However, the visceral fat reducing agent described in Patent Document 1 requires complicated processes to manufacture, such as preparing a culture medium, picking and culturing the Cordyceps militaris. Furthermore, the cultures may contain variations in the content of active ingredients depending on the culture conditions and other factors. As a result, the visceral fat reducing agent may not be stably supplied.

### PRIOR ART DOCUMENT

### Patent Document

Patent Document 1: Japanese Unexamined Patent Publication No. 2020-80708

### DISCLOSURE OF INVENTION

### Means for Solving the Problem

A method for inhibiting body fat accumulation according to the present disclosure includes a process of taking 3.38 mg or more of oenothein B indicated by the following formula (I) as an effective amount per day.

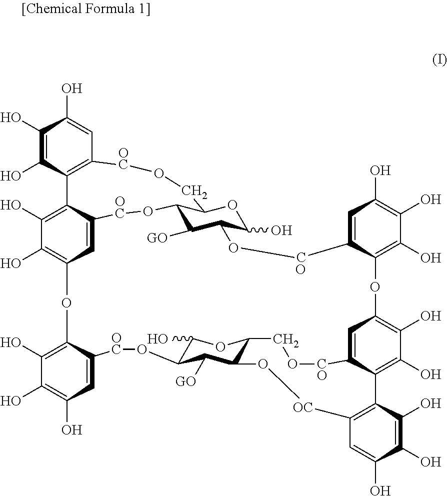

G in the formula (I) represents a structure of the following formula (II).

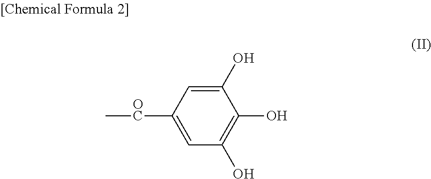

Furthermore, a method for inhibiting body weight gain according to the present disclosure includes a process of taking 3.38 mg or more of oenothein B indicated by the following formula (I) as an effective amount per day.

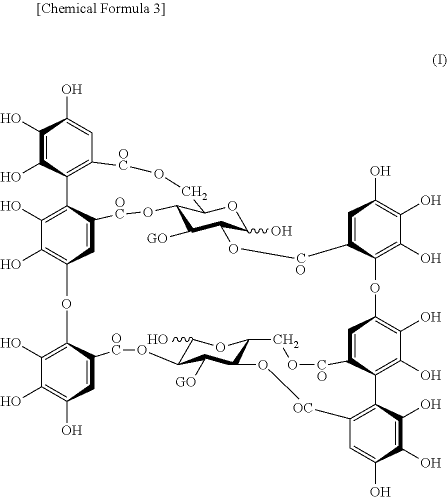

G in the formula (I) represents a structure of the following formula (II).

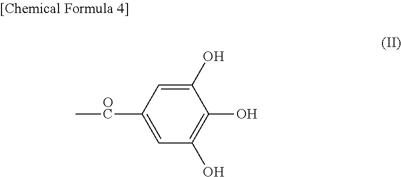

### BRIEF DESCRIPTION OF THE DRAWINGS

FIGS. 1A to 1C are graphs showing test results conducted in Example 1, in which FIG. 1A is a graph showing an amount of change in visceral fat area (VFA), FIG. 1B is a graph showing an amount of change in a body weight, and FIG. 1C is a graph showing an amount of change in body mass index (BMI).

FIGS. 2A and 2B are graphs showing test results conducted in Example 2, in which FIG. 2A is a graph showing an amount of change in VFA, and FIG. 2B is a graph showing an amount of change in liver fat level (CAP: Controlled Attenuation Parameter).

FIG. 3 is a graph showing test results of an amount of change in VFA conducted in Example 3.

FIGS. 4A and 4B are graphs showing test results conducted in Comparative Example 1, in which FIG. 4A is a graph showing an amount of change in a body weight, and FIG. 4B is a graph showing an amount of change in VFA.

### EMBODIMENTS FOR CARRYING OUT THE INVENTION

As described above, in conventional visceral fat reducing agents, the content of active ingredients may vary. Therefore, the visceral fat reducing agents may not be stably supplied. Therefore, there is a need for a method for inhibiting body fat accumulation exhibiting an effect of inhibiting body fat accumulation in a relatively small amount and a method for inhibiting body weight gain exhibiting an effect of inhibiting body weight gain in a relatively small amount, which are derived from natural products, are highly safe, and can be stably taken.

By having the above configuration, the method for inhibiting body fat accumulation according to the present disclosure is derived from a natural product, is highly safe, can be stably taken stably, and exhibits the effect of inhibiting body fat accumulation in a relatively small amount. Furthermore, by having the above configuration, the method for inhibiting body weight gain according to the present disclosure is derived from a natural product, is highly safe, can be stably taken, and exhibits the effect of inhibiting body weight gain in a relatively small amount.

The method for inhibiting body fat accumulation of the present disclosure includes a process of taking oenothein B. Hereinafter, the method for inhibiting body fat accumulation according to an embodiment of the present disclosure will be described.

Oenothein B used in the method for inhibiting body fat accumulation according to one embodiment is a compound belonging to a hydrolyzable tannin and is a type of polyphenol. Oenothein B is represented by the following formula (I). G in the formula (I) represents a structure of the following formula (II).

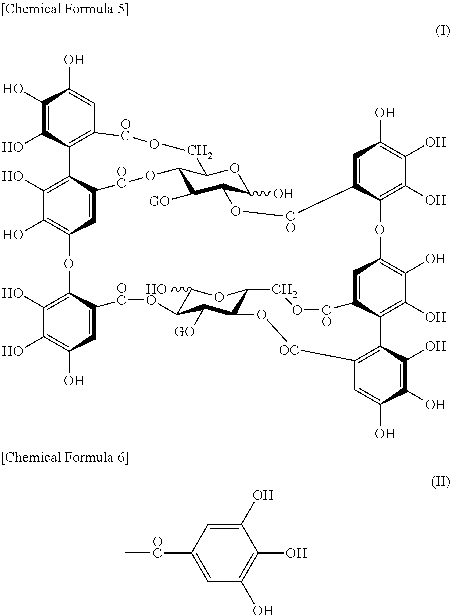

Oenothein B used in the method for inhibiting body fat accumulation according to one embodiment may be derived from a natural product or may be obtained by chemical synthesis. Oenothein B derived from natural products is contained, for example, in archichlamydeae plants in the class Dicotyledoneae of the division Angiospermae (Engler system), which contain hydrolyzable tannin.

Methods for obtaining oenothein B from plants containing hydrolyzable tannin are not limited, and examples thereof include extraction. In the extraction process, plants containing hydrolysable tannins may be used as they are in various parts (whole plants, flowers, calyxes, seeds, fruits, leaves, branches, bark, root bark, rhizomes, roots and the like), or dried. They may be cut, crushed or finely ground as needed.

Examples of the plants containing hydrolyzable tannins include plants that belong to the family Myrtaceae, Rosaceae, Casuarinaceae, Fagaceae, Theaceae, Onagraceae, Lythraceae, Trapaceae, Punicaceae, Melastomataceae, Combretaceae, and Lecythidaceae. These plants contain large amounts of hydrolyzable tannins. Therefore, hydrolyzable tannins can be efficiently obtained by using these plants as a raw material. Among them, plants belonging to the family Myrtaceae are preferred. Furthermore, among the Myrtaceae, many plants in the genus Eucalyptus, Syzygium, Pimenta, and Melaleuca are used for foods, spices, fragrances, and the like. Therefore, it is preferable to use the plants belonging to these groups as a raw material from the viewpoint of food experience and safety. In particular, eucalyptus is preferred because it contains a large amount of oenothein B.

Extraction methods are not limited, and examples thereof include room temperature homogenization extraction, reflux extraction, and supercritical fluid extraction. Extraction using a solvent is performed under conditions that allow hydrolyzable tannin to be eluted. For example, depending on the solvent used, the reaction may be performed for approximately 10 minutes to one week under normal pressure to increased pressure and at a temperature ranging from normal temperature to the boiling point of the solvent. In the case of relatively mild extraction conditions such as normal temperature and normal pressure, it is preferable to immerse the plant containing hydrolyzable tannin in the extraction solvent for a relatively long time (for example, approximately one week). On the other hand, in the case of relatively harsh extraction conditions such as high temperature and increased pressure, it is preferable to complete the extraction process in a relatively short time (for example, 10 minutes or more and 3 hours or less).

Examples of the solvent used for extraction include those commonly used depending on plant species or treatment process. Specifically, such solvents include water; alcohols (for example, lower alcohols such as methanol and ethanol, and polyhydric alcohols such as ethylene glycol, propylene glycol, 1,3-butylene glycol, and glycerin); ketones such as acetone and other ketones with relatively high polarity; and organic solvents such as esters such as ethyl acetate. Extraction solvents may be used alone or in combination of two or more. Considering that the body fat accumulation inhibitor according to one embodiment is to be taken into the body, water, ethanol, or aqueous ethanol are preferred among these.

A form of use after extraction is not particularly limited, and the extract containing oenothein B may be used as it is, or may be processed by drying, spray drying or the like after concentration. It may be purified if necessary. In the case of purification, complicated processes are required to isolate oenothein B, and it is also disadvantageous in terms of cost. Therefore, the purification may not be a purification that isolates oenothein B, but a purification that increases the concentration of oenothein B contained in the extract by removing only extract components that are relatively easy to remove. For such purification, for example, an adsorption resin such as Diaion HP20 (manufactured by Mitsubishi Chemical Corporation) is used. For example, an extract containing oenothein B in the extract component in a proportion of preferably 0.5% by mass or more, more preferably 18 by mass or more may be used. The extract with an increased concentration of oenothein B can be used in a smaller amount than the unpurified extract.

In the method for inhibiting body fat accumulation according to one embodiment, in order to exhibit the effect of inhibiting body fat accumulation, it is necessary to take 3.38 mg or more of oenothein B as an effective amount per day. When the extract is used, it may be used so that the effective amount of oenothein B per day is 3.38 mg or more. If the daily intake of oenothein B is less than 3.38 mg, the effect of inhibiting body fat accumulation will not be sufficiently demonstrated. On the other hand, the daily intake of oenothein B may be 8.12 mg or less. Although there is no safety or other problems when taking more than 8.12 mg, for example, when it is mixed in food and beverages, it may affect the flavor of some food and beverage products.

The method for inhibiting body fat accumulation according to one embodiment efficiently inhibits accumulation of visceral fat and liver fat among body fat. The visceral fat is fat that accumulates mainly around organs such as intestines and stomach. The liver fat is fat that accumulates in liver (hepatocytes). Accumulation of the visceral fat and the liver fat causes health problems such as dyslipidemia, diabetes, hypertension, fatty liver and vascular diseases. The liver fat, in particular, is known to cause non-alcoholic steatohepatitis (NASH) in severe cases, which can progress from cirrhosis to liver cancer.

In the method for inhibiting body fat accumulation according to one embodiment, the forms in which oenothein B is taken are not limited, and any form may be used as long as it allows oenothein B to be taken in an effective amount of 3.38 mg or more per day. Specifically, it is sufficient if oenothein B is taken in a processed product that has been processed into a solid, semi-solid (such as paste), or liquid form containing oenothein B by using a pharmaceutically acceptable carrier.

Examples of the pharmaceutically acceptable carriers include excipients, binders, disintegrants, lubricants, preservatives, stabilizers, buffers, suspending agents, and surfactants. Specifically, solid carriers (such as starch, dextrin, sugars, lactose, mannitol, processed starch, gelatin, cellulose, carboxymethylcellulose, hydroxypropylmethylcellulose, casein, organic acids and inorganic salts), liquid carriers (such as distilled water, physiological saline, glucose aqueous solution, fructose glucose liquid sugar, high fructose liquid sugar, ethanol, propylene glycol, polyethylene glycol, and glycerin), and oil carriers (such as various animal and vegetable oils, white petrolatum, paraffin, and wax).

Examples of the processed products containing oenothein B include oral administration agents such as tablets, capsules, pills, granules, powders, emulsions, suspensions, and syrups. In addition to such oral administration agents, it is conceivable that oenothein B may be incorporated into various processed foods such as prepared foods, retort foods, canned goods, breads, confectionery (such as cookies, chocolates, candies, gummies, and rice crackers), dairy products (such as yogurt and milk-based drinks), and beverages (such as tea-based drinks and soft drinks), sauces and soups so that 3.38 mg or more of oenothein B can be taken per day. The processed product containing oenothein B may be, for example, in the form of a daily unit package. When the processed product containing oenothein B is in the form of a daily unit package, an effective amount of oenothein B per day can be easily taken by simply taking one package per day.

The method for inhibiting body fat accumulation according to one embodiment, which includes a process of taking 3.38 mg or more of oenothein B as an effective amount per day, exhibits a body fat accumulation inhibiting effect. Specifically, the method for inhibiting body fat accumulation according to one embodiment reduces body fat can significantly inhibit accumulation of body fat, particularly visceral fat and liver fat, by continuously taking oenothein B for 4 weeks or more, compared to the case where no intake is made.

In this way, the method for inhibiting body fat accumulation according to one embodiment exhibits the effect of inhibiting accumulation of body fat such as visceral fat and liver fat. As a result, the effect of inhibiting body weight gain is also exhibited. That is, a method for inhibiting body weight gain according to the present disclosure includes a process of taking 3.38 mg or more of oenothein B as an effective amount per day.

### EXAMPLES

Hereinafter, the present disclosure will be specifically described with reference to Examples and Comparative Examples. However, the present disclosure is not limited to these examples.

### Preparation Example 1

Oenothein B was prepared by using the following procedure. Eucalyptus leaves were extracted with aqueous ethanol, filtered and concentrated, and then dextrin was added as an excipient. Then, a powdered extract containing oenothein B as a main component was obtained by spray drying.

### <Test Food 1>

Test Food 1, containing the powdered extract obtained in Preparation Example 1, gelatin, cellulose, dextrin, starch, calcium stearate, caramel coloring, plant lecithin, and talc, was prepared by using a capsule encapsulated with gelatin and colored brown with caramel coloring.

### <Placebo Food 1>

Placebo Food 1 was prepared in the same manner as Test Food 1 except that the powdered extract obtained in Preparation Example 1 was not used.

Components of Test Food 1 and Placebo Food 1 were analyzed by using a high performance liquid chromatography. The amount of oenothein B in 3 capsules (for one day) of Test Food 1 was 3.38 mg. On the other hand, the amount of oenothein B in Placebo Food 1 in 3 capsules (for one day) was 0 mg. Test Food 1 and Placebo Food 1 were indistinguishable in appearance and flavor.

### Example 1

A randomized, placebo-controlled, double-blind, parallel study was conducted with healthy men and women, aged 20 or more and less than 65 and with BMI of 23 kg/m² to 30 kg/m², as subjects. An identification number was assigned to a person responsible for allocation by a test food sender. The person responsible for allocation used a computer to generate random numbers and created an allocation table by using a completely randomized design with defined variables as factors.

The subjects were randomly assigned to either a test food group or a placebo group based on stratified random assignment considering VFA, BMI, age, gender and the like. The allocation table was disclosed only to the test food sender. The allocation table was kept locked by the person responsible for allocation until the day of key opening. Test-related persons were not informed of the allocation of each group and were not involved in the allocation process.

The subjects took 3 capsules (for one day) of Test Food 1 or Placebo Food 1 with water or lukewarm water every day just before a snack after 2 pm or dinner for 12 weeks. Those with a capsule intake rate of 80% or higher were defined as subjects for data analysis. The height was measured only at the first examination. The first examination was used as a baseline, and a body weight and a tomography of a cross section of the umbilical part by a Computed Tomography (CT) were measured every 4 weeks. The statistical significance level was set at 5% with a two-tailed test.

Data were expressed as mean value±standard deviation for subject backgrounds and mean value±standard error for evaluation items. Data at each time point and changes from the baseline were compared between the groups by time in a linear mixed model with baseline values as covariates and time, groups and interaction between time and groups, interaction between baseline values and time, and test subjects as factors at each time point.

Of the 198 subjects, 189 were accepted as subjects for data analysis. The test food group (94 subjects) consisted of 45 males and 49 females with the mean age of 46.5±10.7 years, height of 164.4±8.5 cm, body weight of 69.6±8.9 (kg) and VFA of 99.6±38.9 (cm²). On the other hand, the placebo food group (95 subjects) consisted of 45 males and 50 females with the mean age of 48±10 years, height of 164.6±8.9 cm, body weight of 69.6±9.2 (kg), and VFA of 99.3±36.5 (cm²).

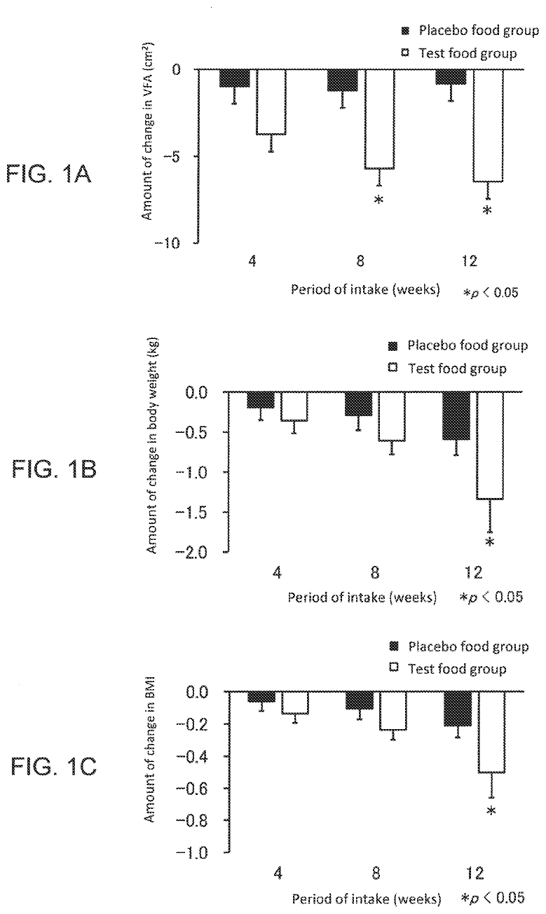

As a result of the comparison between the groups, the test food group had significantly lower VFA after 8 and 12 weeks (FIG. 1A), and significantly lower body weight gain (FIG. 1B) and BMI (FIG. 1C) after 12 weeks than the placebo food group. From the above, it was confirmed that the intake of the test food inhibited VFA, body weight gain, and BMI. No adverse events caused by the test food were confirmed.

### Example 2

A randomized, placebo-controlled, double-blind, parallel study was conducted with healthy men, aged 20 or more to less than 60 and with BMI of 23 kg/m² to 30 kg/m², as subjects. An identification number was assigned to a person responsible for allocation by a test food sender. The person responsible for allocation used a computer to generate random numbers and created an allocation table by using a completely randomized design with defined variables as factors.

The subjects were randomly assigned to either a test food group or a placebo group based on stratified random assignment considering VFA, CAP, BMI, age and the like. The allocation table was disclosed only to the test food sender. The allocation table was kept locked by the person responsible for allocation until the day of key opening. Test-related persons were not informed of the allocation of each group and were not involved in the allocation process.

The subjects took 3 capsules (for one day) of Test Food 1 or Placebo Food 1 with a beverage containing 18 g sucrose at snack time after 3 pm or dinner for 12 weeks every day. Those with a capsule intake rate of 85% or higher were defined as subjects for data analysis. The height was measured only at the first examination. The first examination was used as a baseline, and VFA of the umbilical part performed by a dual impedance method by using a DualScan HDS-2000 of Omron Corporation, and CAP performed by an ultrasound elastography method by using a Fibroscan 530 Compact of Integral Corporation were measured every 6 weeks. The statistical significance level was set at 5% with a two-tailed test.

Data were expressed as mean value±standard deviation for subject backgrounds and mean value±standard error for evaluation items, and comparisons within the groups were performed by using a Dunnett's multiple test. Of the 40 subjects, 38 were accepted as subjects for data analysis. The mean age of the test food group (18 subjects) was 47±10 years, with the height of 172.5±4.1 cm, VFA of 95.1±17.0 (cm²) and CAP of 292±48 (dB/m). On the other hand, the mean age of the placebo food group (20 subjects) was 46±9 years, with the mean height of 170.9±6.0 cm, VFA of 94.6±14.1 (cm²), and CAP of 276±46 (dB/m).

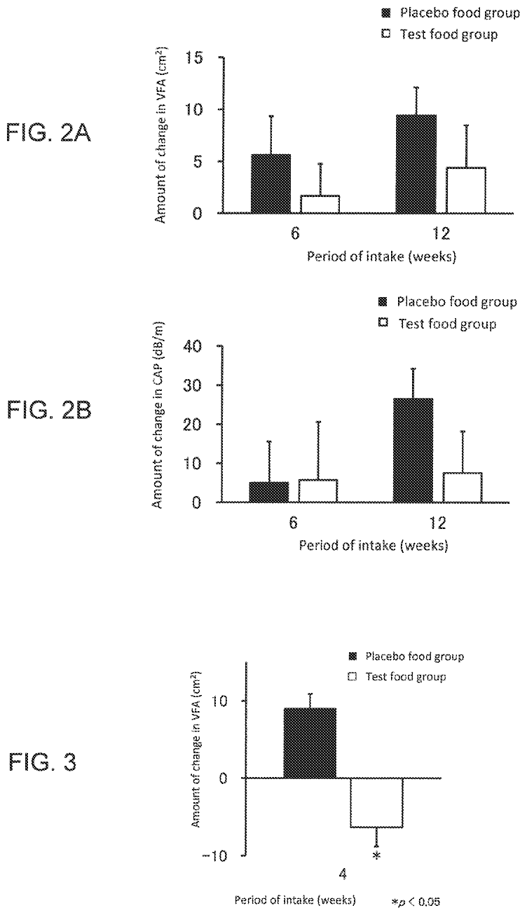

When each group was compared within the group, VFA (p<0.01) and CAP (p<0.05) after 12 weeks showed a significant increase in the placebo food group. On the other hand, no significant changes were observed in the test food group, and an effect of inhibiting the increase in VFA and CAP due to intake of the test food was observed (FIG. 2). No adverse events caused by the test food were confirmed.

### <Test Food 2>

Test Food 2 was prepared in the same manner as Test Food 1 except that the powdered extract obtained in Preparation Example 1 was used to set oenothein B contained in 6 capsules (for one day) to be 8.12 mg. The content of oenothein B was measured by using a high performance liquid chromatography.

### <Placebo Food 2>

Placebo Food 2 was prepared in the same manner as Test Food 2 except that the powdered extract obtained in Preparation Example 1 was not used. As a result of component analysis using a high performance liquid chromatography, the amount of oenothein B contained in 6 capsules (for one day) of Placebo Food 1 was 0 mg. Test Food 2 and Placebo Food 2 were indistinguishable in appearance and flavor.

### Example 3

A randomized, placebo-controlled, double-blind, crossover study was conducted with healthy men aged 20 or more as subjects. An identification number was assigned to a person responsible for allocation by a test food sender. The person responsible for allocation used a computer to generate random numbers and created an allocation table by using a completely randomized design with defined variables as factors.

The subjects were randomly assigned to either a test food group or a placebo group based on stratified random assignment considering VFA, BMI, age and the like. The allocation table was disclosed only to the test food sender. The allocation table was kept locked by the person responsible for allocation until the day of key opening. Test-related persons were not informed of the allocation of each group and were not involved in the allocation process.

The subjects took 3 capsules each, total 6 capsules (for one day) of Test Food 2 or Placebo Food 2 with water or lukewarm water at 3 μm and 9 pm every day for 4 weeks. Immediately after taking the 3 capsules, they consumed a food or drink containing 20 g of sugar in terms of sucrose (total of 40 g per day in terms of sucrose). Those with a capsule intake rate of 85% or higher were defined as subjects for data analysis. The height was measured only at the examination before the intake. Visceral fat area in the umbilical part was measured by a dual-scan method before the start of intake and after the end of intake. After a 4-week rest period, Test Food 2 and Placebo Food 2 taken by the subjects were replaced, and the intake test for 4 weeks was conducted again. The statistical significance level was set at 5% using a two-tailed test.

Data were expressed as mean value±standard deviation for subject backgrounds and mean value±standard error for evaluation items, and comparisons between the groups and within the group were performed by using Student's t-test. All 6 subjects were accepted as subjects for data analysis. The mean age of the test food group was 47±12 years, with the height of 170.7±6.7 cm. VFA at the beginning of the test food intake was 88.5±11.1 (cm²), and VFA at the beginning of the placebo intake was 78.2±10.8 (cm²).

The comparison between the groups showed that the test food group had significantly lower VFA after 4 weeks than the placebo food group (FIG. 3). The comparison within the group showed that the placebo food group had a significant increase in VFA (p<0.005). On the other hand, there was a significant decrease in the test food group (p<0.05), indicating that the intake of the test food inhibited VFA. No adverse events caused by the test food were confirmed.

### <Test Food 3>

Test Food 3 was prepared in the same manner as Test Food 1 except that the powdered extract obtained in Preparation Example 1 was used to set oenothein B contained in 6 capsules (for one day) to be 1.12 mg. The content of oenothein B was measured by using a high performance liquid chromatography.

### <Placebo Food 3>

Placebo Food 3 was prepared in the same manner as Test Food 3 except that the powdered extract obtained in Preparation Example 1 was not used. As a result of component analysis using a high performance liquid chromatography, the amount of oenothein B contained in 6 capsules (for one day) of Placebo Food 3 was 0 mg. Test Food 3 and Placebo Food 3 were indistinguishable in appearance and flavor.

### Comparison Example 1

A randomized, placebo-controlled, double-blind, parallel study was conducted with healthy men and women, aged 20 or more and with BMI of 23 kg/m² to 30 kg/m², as subjects. An identification number was assigned to a person responsible for allocation by a test food sender. The person responsible for allocation used a computer to generate random numbers and created an allocation table by using a completely randomized design with defined variables as factors.

The subjects were randomly assigned to either a test food group or a placebo group based on stratified random assignment considering VFA, BMI, age, gender and the like. The allocation table was disclosed only to the test food sender. The allocation table was kept locked by the person responsible for allocation until the day of key opening. Test-related persons were not informed of the allocation of each group and were not involved in the allocation process.

The subjects took 6 capsules (for one day) of Test Food 3 or Placebo Food 3 with water or lukewarm water every day just before a snack after 2 pm or dinner for 12 weeks. Those with a capsule intake rate of 80% or higher were defined as subjects for data analysis. The height was measured only at the first examination. The first examination was used as a baseline, and visceral fat area in the umbilical part was measured every 6 weeks by using a dual-scan method. The statistical significance level was set at 5% with a two-tailed test.

Data were expressed as mean value±standard deviation for subject backgrounds and mean value±standard error for evaluation items, and comparisons between the groups were performed by using a Student's t-test and comparisons within the group were performed by using a Dunnett's multiple test. All 20 subjects were accepted as subjects for data analysis. The test food group (10 subjects) consisted of 4 males and 6 females, with the mean age of 51.5±9.5 years, height of 162.4±9.9 cm, and body weight of 69.2±9.1 (kg), and VFA was 78.4±22.5 (cm²). On the other hand, the placebo food group (10 subjects) consisted of 4 males and 6 females with the mean age of 51.1±7.8 years, height of 161.3±5.8 cm, body weight of 68.3±6.1 (kg), and VFA of 75.2±21.4 (cm²).

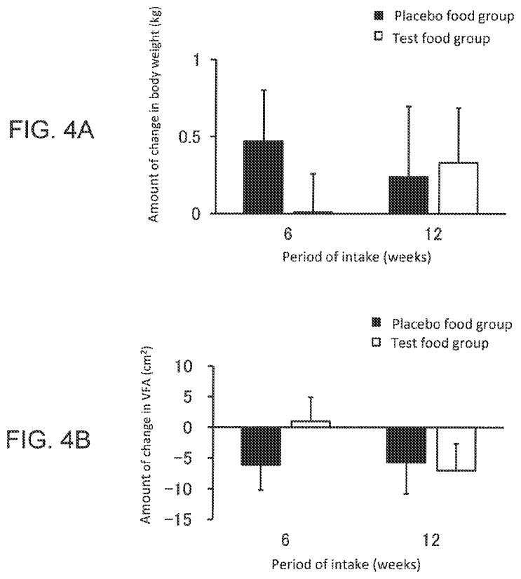

In both comparisons between the groups and within the group, no significant changes were observed in a body weight or VFA, and no effect of test food intake was observed (FIG. 4). No adverse events caused by the test food were confirmed.

1. A method for inhibiting body fat accumulation comprising a process of taking 3.38 mg or more of oenothein B indicated by the following formula (I) as an effective amount per day. 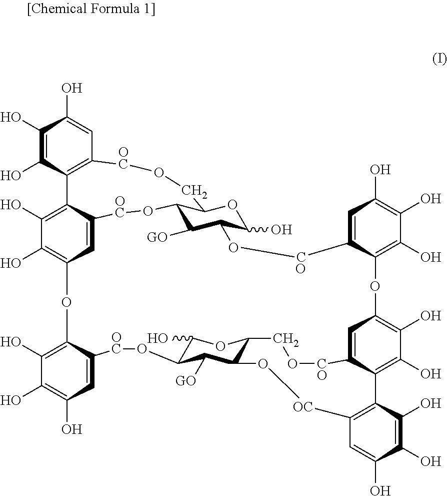
G in the formula (I) represents a structure of the following formula (II).

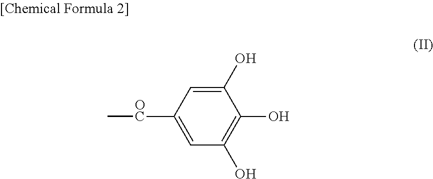

2. The method for inhibiting body fat accumulation according to claim 1 comprising extract of eucalyptus plant containing the oenothein B.

3. The method for inhibiting body fat accumulation according to claim 1, wherein body fat is visceral fat.

4. The method for inhibiting body fat accumulation according to claim 1, wherein body fat is liver fat.

5. The method for inhibiting body fat accumulation according to claim 1 being taken in the form of a daily unit package.

6. The method for inhibiting body fat accumulation according to claim 2, wherein body fat is visceral fat.

7. The method for inhibiting body fat accumulation according to claim 2, wherein body fat is liver fat.

8. The method for inhibiting body fat accumulation according to claim 2 being taken in the form of a daily unit package.

9. A method for inhibiting body weight gain comprising a process of taking 3.38 mg or more of oenothein B indicated by the following formula (I) as an effective amount per day. 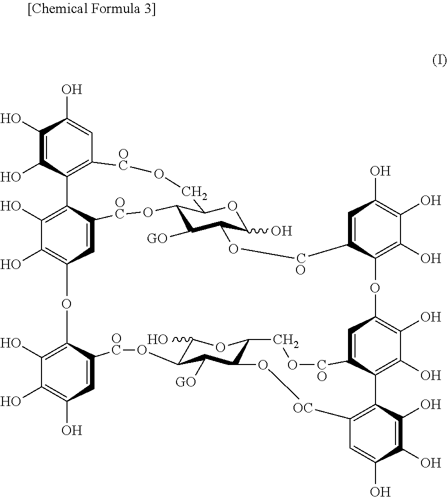
G in the formula (I) represents a structure of the following formula (II).

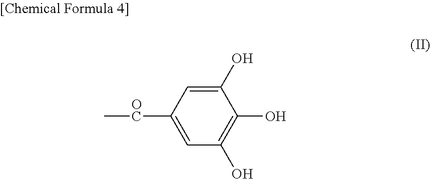

10. The method for inhibiting body weight gain according to claim 9 comprising extract of eucalyptus plant containing the oenothein B.

11. The method for inhibiting body weight gain according to claim 9 being taken in the form of a daily unit package.

12. The method for inhibiting body weight gain according to claim 10 being taken in the form of a daily unit package.
